(19) United States
(12) Patent Application Publication
(10) Pub. No.: US 2024/0416326 A1
(43) Pub. Date: Dec. 19, 2024

(54) NICKEL PROMOTED COPPER ALUMINA CATALYST FOR SLURRY PHASE PROCESS FOR PRODUCING FATTY ALCOHOL

(71) Applicant: BASF CORPORATION, Florham Park, NJ (US)

(72) Inventors: Arunabha KUNDU, Beachwood, OH (US); Scott HEDRICK, Beachwood, OH (US); Noemi N. TRENT, Beachwood, OH (US)

(73) Assignee: BASF CORPORATION, Florham Park, NJ (US)

(21) Appl. No.: 18/840,738

(22) PCT Filed: Feb. 17, 2023
(86) PCT No.: PCT/US2023/013273

§ 371 Date: Aug. 22, 2024

Related U.S. Application Data
(60) Provisional application No. 63/313,461, filed on Feb. 24, 2022.

(51) Int. Cl. B01J 23/755; B01J 35/30; B01J 35/40; B01J 35/61; B01J 37/00; B01J 37/03; B01J 37/04; B01J 37/08; C07C 29/154

(52) U.S. Cl. CPC B01J 23/755; B01J 35/394; B01J 35/40; B01J 35/613; B01J 37/009; B01J 37/038; B01J 37/04; B01J 37/088; C07C 29/154

(57) ABSTRACT

Described herein is a catalyst including a copper source, a nickel source and alumina that does not include chromium to address regulatory concerns in the industry. The catalyst has a Brunauer-Emmet-Teller surface area of about 20 m²/g to about 50 m²/g. A method of preparing a catalyst is also described herein.

### CROSS REFERENCE TO RELATED APPLICATION(S)

The present application claims priority to U.S. Provisional Patent Application No. 63/313,461 filed on Feb. 24, 2022, the entire contents of which are incorporated in its entirety.

### FIELD OF THE INVENTION

The present invention relates generally to the field of catalysts for use in a hydrogenolysis or hydrogenation. More specifically, the present invention is related to a nickel (Ni) promoted copper (Cu) alumina powder catalyst for use in a slurry phase process for producing fatty alcohol.

### BACKGROUND OF THE INVENTION

In commercial slurry processes for producing fatty alcohols, copper-chromium (CuCr) catalysts are typically employed. CuCr catalysts have been used for their high performance and better stability. However, chrome containing catalysts are considered hazardous chemicals that impact human health and pollute the environment under the REACH regulation. In accordance with the REACH regulation, the substance Chromium (VI) trioxide (CrO₃) cannot be used in the European Union. Furthermore, it has been found that the CuCr catalyst may potentially contain trace Cr (6+) as an impurity, which is carcinogenic.

Thus, a need exists to find a Cr-free catalyst powder for use in a slurry phase fatty alcohol system. But this has been very challenging as two parameters, good performance and high attrition resistance (both chemically and mechanically), need to work simultaneously. During the downstream process of filtration or centrifugation, the production of fines (less than 1 micron) may be unacceptable even if the catalyst has very good performance. Thus, a powder catalyst with good particle size distribution and stability leads to better liquid product separation by filtration or centrifugation from the catalyst slurry.

### SUMMARY OF THE INVENTION

It has been found that including nickel in the catalyst aids in keeping the surface area of the catalyst higher, while also allowing the catalyst to be processed at a higher temperature, i.e. from about 850° C. to about 900° C. Ni also helps to maintain the hydrogenation activity and the chemical stability under reaction conditions.

In one embodiment of the present disclosure, a catalyst is provided including a copper source, a nickel source, and alumina, wherein the catalyst is substantially free of chromium. In some embodiments, the catalyst does not include chromium.

In some embodiments, the catalyst of the present disclosure may have an average particle size of about 8 μm to about 12 μm. In other embodiments, the catalyst may have an average particle size of about 5 μm to about 20 μm.

In some embodiments, the catalyst may include the copper source m an amount of about 25 wt % to about 50 wt %, or about 30 wt % to about 40 wt % based on a total weight of the catalyst.

In some embodiments, the catalyst may include the nickel source in an amount of about 2 wt % to about 12 wt %, or about 2 wt % to about 6 wt % based on a total weight of the catalyst.

In some embodiments, the catalyst may have a Brunauer-Emmett-Teller (“BET”) surface area of about 20 m²/g to about 50 m²/g.

In some embodiments, the copper source of the catalyst may include copper oxide, copper nitrate, copper sulfate, copper chloride, copper bromide, copper fluoride, copper acetate, copper carbonate, or a combination of any two or more thereof. In another embodiment, the copper source may be copper nitrate.

In some embodiments, the nickel source of the catalyst may include nickel sulfate, nickel chloride, nickel bromide, nickel acetate, nickel oxide, nickel nitrate or a combination of any two or more thereof. In another embodiment, the nickel source may be nickel nitrate.

In some embodiments, the alumina may include aluminum nitrate, sodium aluminate or powder alumina.

In another embodiment, a method of preparing a catalyst may include mixing a nickel precursor and alumina in a solution; adding the solution to a copper precursor and mixing to form a second solution; adding a caustic material to the second solution to form an aqueous slurry including a precipitate; collecting the precipitate; drying the precipitate to form a dried precipitate, and calcining the dried precipitate to form a catalyst, wherein the calcining is conducted at a temperature of about 850° C. to about 900° C., and wherein the catalyst has a Brunauer-Emmett-Teller (“BET”) surface area of about 15 m²/g to about 50 m²% g.

In some embodiments, the aqueous slurry may be at a pH of about 7.0 to about 8.0, or about 8.0.

In some embodiments of the method, the collecting may include filtering the aqueous slurry to remove the precipitate. In some embodiments of the method, the calcining may be conducted for about 1 hour to about 4 hours.

In some embodiments of the method, the cooper precursor may be a copper salt including copper nitrate, copper sulfate, copper chloride, copper fluoride, copper bromide, copper acetate, or a combination of any two or more thereof. In some embodiments of the method, the nickel precursor may include nickel nitrate or nickel oxide. In some embodiments, the alumina may be aluminum nitrate, sodium aluminate or powder alumina.

In some embodiments of the method, wherein the caustic source may be sodium carbonate, sodium hydroxide, potassium carbonate, potassium hydroxide, or a combination of any two or more thereof.

In some embodiments of the method, the catalyst may be substantially free of chromium. In another embodiment of the method, the catalyst does not include chromium.

In another embodiment, a method of hydrogenating a carbonyl-containing organic compound, the method includes contacting the carbonyl-containing organic compound with a catalyst of the present disclosure.

### BRIEF DESCRIPTION OF THE DRAWINGS

FIG. 1 is a graph illustrating the particle size distribution during precipitation according to Example 1.

FIG. 2 is an XRD pattern of the catalyst of Example 1.

FIG. 3 is an XRD pattern of the catalyst of Example 2.

FIG. 4 is an XRD pattern of the catalyst of Example 3.

FIG. 5 is an XRD pattern of the catalyst of Example 4.

FIG. 6 is a schematic diagram of an autoclave reactor used for catalyst performance testing, according to various embodiments.

FIG. 7 is a graphical comparison of fatty alcohol yield in slurry phase methyl ester hydrogenolysis process over time for a commercial CuCr catalyst to the catalysts of Examples 1-4.

FIG. 8 is a graph illustrating the particle size distribution of the fresh catalyst powder of Examples 1-4.

FIG. 9 is a graph illustrating the particle size distribution after reaction of Example 2.

### DETAILED DESCRIPTION OF THE INVENTION

The present invention advances the state of the art by developing a nickel (Ni) promoted copper (Cu) alumina powder catalyst. This catalyst is processed at a very high temperature for use in a slurry phase fatty alcohol process. Without being limited to a theory, the inventors believe that nickel promotion helps to give high hydrogenation activity and good attrition/chemical resistance. The high-temperature processing also makes it better with respect to strength as nickel interacts with copper and aluminum to give a spinel phase. Thus, the catalyst of the present disclosure is not only good in terms of performance in slurry phase fatty alcohol processes, but can also maintain its integrity during downstream processes (filtration or centrifugation).

The catalyst of the present disclosure may be good in several slurry phase fatty alcohol processes, for example in slurry phase methyl ester hydrogenolysis, wax ester hydrogenolysis, or styrene monomer/propylene oxide (SMPO) slurry process. In other embodiments, tablets or extrudates may be produced from the catalyst powder of the present disclosure. A tablet or extrudate may be used for fixed-bed hydrogenation processes, such as hydrogenolysis of fatty acid methyl ester and fatty acid wax ester and oxo-aldehyde hydrogenation.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term

All references to wt % throughout the specifications and the claims refer to the weight of the component in reference to the weight of the entire composition and may also be designated as w/w.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein, “substantially free” is intended to indicate that, to the extent possible, the material being described is excluded from the formulation. However, trace amounts may be carried through due to contamination in starting reagents. For example, where the term used is “substantially free of chromium,” it is intended that all chromium is to be ideally excluded, however, trace amounts of chromium may be carried due to contamination by chromium of the other starting reagents such as the copper, manganese, and aluminum source materials. For example, in some embodiments, “substantially free of chromium” may include less than 1000 ppm chromium, such as less than 750 ppm chromium, less than 500 ppm chromium, or less than 100 ppm chromium. Where appropriate, substantially free of chromium means that the catalyst contains no detectable chromium (0.0 wt % chromium). Similarly, the term is also applied to manganese, in some embodiments.

The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

An object of the present disclosure is to prepare a catalyst that does not contain chromium. In one embodiment, the catalyst of the present disclosure does not contain chromium. The catalyst of the present disclosure has comparable activity to current commercially used chromium containing catalysts. Further, the catalyst of the present disclosure also has a comparable fatty alcohol yield when compared to the commercial chromium catalyst. It has also been found that the catalysts of the present disclosure may have a broad range of copper oxide, nickel oxide and aluminum oxide that show good performance to produce fatty alcohol.

The catalyst of the present disclosure may be a copper, nickel and alumina catalyst (Cu—Ni—Al). In one embodiment of the present disclosure, a catalyst includes a copper source, a nickel source and alumina. In some embodiments, the catalyst may be a powder. The catalyst of the present disclosure is free of chromium. In some embodiments, the catalyst may include about 25 wt % to about 50 wt % of copper oxide, about 1 wt % to about 12 wt % of nickel oxide and the remaining amount being alumina.

The copper source of the present disclosure may include copper oxide, copper nitrate, copper sulfate, copper chloride, copper bromide, copper fluoride, copper acetate, copper carbonate or a combination of any two or more thereof. In one embodiment, the copper source may be copper oxide or copper nitrate. In another embodiment, the copper source may be copper oxide. In an additional embodiment, the copper source may be copper nitrate.

The nickel source of the present disclosure may include nickel sulfate, nickel chloride, nickel bromide, nickel acetate, nickel oxide, nickel nitrate, or a combination of any two or more thereof. In one embodiment, the nickel source may be nickel oxide. In another embodiment, the nickel source may be nickel nitrate.

In some embodiments, the alumina may be aluminum nitrate, sodium aluminate or powder alumina.

In some embodiments, the catalyst may include copper oxide in an amount from about 20 wt %, about 25 wt %, about 30 wt %, or about 35 wt % to about 55 wt %, about 50 wt %, about 45 wt %, or about 40 wt %, based on the total weight of the catalyst. In other embodiments, the catalyst may include copper oxide in an amount from about 20 wt % to about 55 wt %, about 25 wt % to about 50 wt %, about 30 wt % to about 45 wt %, or about 35 wt % to about 30 wt %, based on total weight of the catalyst.

In some embodiments, the catalyst may include nickel oxide in an amount from about 1 wt %, about 2 wt %, about 3 wt %, about 4 wt %, or about 5 wt %, to about 6 wt %, about 7 wt %, about 8 wt %, about 9 wt %, about 10 wt %, about 11 wt %, or about 12 wt %, based on the total weight of the catalyst. In other embodiments, the catalyst may include nickel oxide in an amount from about 1 wt % to about 12 wt %, about 2 wt % to about 11 wt %, about 3 wt % to about 10 wt %, about 4 wt % to about 9 wt %, about 5 wt % to about 8 wt %, or about 6 wt % to about 7 wt %, based on the total weight of the catalyst.

In some embodiments, the catalyst may include alumina in an amount from about 33 wt % to about 79 wt %, based on the total weight of the catalyst. In another embodiment, the catalyst may include copper oxide and nickel oxide in an amount described above, and the alumina is included in a remaining amount to total 100 wt % of the catalyst.

In one embodiment of the present disclosure, the catalyst does not include chromium. In another embodiment of the present disclosure, the catalyst is substantially free of chromium.

The catalyst of the present disclosure may be used for hydrogenation or hydrogenolysis in a slurry phase. For example, the catalyst may be used in slurry phase methyl ester hydrogenolysis, wax ester hydrogenolysis or SMPO slurry process.

In some embodiments, the catalyst of the present disclosure may have a Brunauer-Emmett-Teller (“BET”) surface area (“SA”) from about 20 m²/g to 50 m²/g, from about 25 m²/g to about 45 m²/g, from about 20 m²/g to about 40 m²/g, or from about 25 m²/g to about m²/g. In other embodiments, the catalyst of the present disclosure may have a BET surface are of about 20 m²/g, about 25 m²/g, about 30 m²/g, about 35 m²/g, about 40 m²/g, about 45 m²/g, or about 50 m²/g.

The catalyst of the present disclosure exhibits a spinel copper aluminate with a small amount of copper oxide, which provides more stability, formed after calcining at higher temperatures of about 800° C. to about 900° C. In some embodiments, the calcining may be at a temperature of about 800° C., about 810° C., about 820° C., about 830° C., about 840° C., about 850° C., about 860° C., about 870° C., about 880° C., about 890° C., or about 900° C.

In one embodiment, the catalyst of the present disclosure may be a Cu—Ni—Al catalyst. The Cu—Ni—Al catalyst may be for methyl ester hydrogenolysis and have mainly a CuAl₂O₄ crystal phase and a small amount of CuO. The Cu—Ni—Al catalyst may have a BET surface area from about 20 m²/g to about 50 m²/g.

The Cu—Ni—Al catalyst may be in the form of a powder. An average particle size of the powder may be described according to the following particle size distribution (“PSD”) D₁₀ about 1 μm to about 10 μm, D₅₀ from about 10 μm to about 25 μm microns, and D₉₀ from about 40 μm to about 75 μm. This may include a PSD of: D₁₀ about 2 μm to about 3.5 μm, D₅₀ from about 16 μm to about 21 μm microns, and D₉₀ from about 54 μm to about 75 μm.

The Cu—Ni—Al catalyst may also have an average particle size of from about 5 μm to about 20 μm, or from about 8 μm to about 12 μm. In other embodiments, the Cu—Ni—Al catalyst may have an average particle size of about 5 μm, about 8 μm, about 12 μm, about 15 μm, or about 20 μm.

The filtration properties are also important in fatty alcohol production when using slurry phase processes because the catalysts must be separated from the reactor slurry for reuse, and to allow for pure fatty alcohol products. The catalyst of the present disclosure exhibits good filtration properties that are similar to those of the Cu-chromium commercial catalysts. A catalyst with good filtration/separation properties will enable the fatty alcohol producing plant a high production throughput.

In another embodiment of the present disclosure, a method of preparing a catalyst is provided. The method includes mixing a nickel precursor and alumina in a solution with water, and adding a copper precursor, and mixing again. The mixture is pumped to a vessel with a certain amount of water heel. A caustic material is then pumped to the vessel to form an aqueous slurry comprising a precipitate at a certain pH, collecting the precipitate, which is subsequently separated from the slurry. The precipitate is then dried and calcined to form the catalyst. The catalyst does not contain chromium. In another embodiment, the catalyst may be substantially free of chromium.

In some embodiments, the collection of the precipitate may be via filtration of the aqueous slurry to remove and collect the precipitate as a filter cake. The precipitate may also be washed with deionized (“DI”) water to remove some of the sodium from the filter cake. The washing may be conducted with large volumes of water and may repeated two, three, four or more times.

In the method of preparation, the drying of the precipitate may be done in an oven in a heated atmosphere. The heating may be from about 40° C. to about 200° C., from about 75° C. to about 150° C., or from about 100° C. to about 125° C. In some embodiments, the heating may be about 40° C., about 60° C. about 80° C., about 100° C. about 120° C. about 140° C., about 160° C., about 180° C., or about 200° C. The drying may be done for a time period to ensure a dried powder. According to various embodiments, the time period may be from 1 hour to 24 hours, or more. This includes about 5 hours to about 15 hours, or about 8 hours to about 12 hours. In some embodiments, the timer period for drying may be about 1 hour, about 3 hours, 5 hours, about 8 hours, about 12 hours, about 15 hours, about 18 hours, about 20 hours, or about 24 hours. In some embodiments, the drying is overnight.

In the method, the calcining may be conducted at a temperature from about 750° C. to about 1200° C., from about 800° C. to about 1100° C., or from about 900° C. to about 1000° C. In one embodiment, the catalyst may be processed at a temperature of about 850° C. to about 900° C. In another embodiment, the catalyst may be processed at a temperature of about 750° C., about 800° C., about 850° C., about 900° C., about 950° C., about 1000° C., about 1050° C., about 1100° C., about 1150° C., or about 1200° C. The calcining may be done for a time period to complete calcination of the dried powder. According to various embodiments, the time period may be for about 10 minutes to about 10 hours. This includes about 10 minutes to about 10 hours, about 30 minutes to about 9 hours, 1 hour to about 4 hours, about 2 hours to about 8 hours, about 3 hours to about 7 hours, or about 4 hours to about 6 hours.

In any of the embodiments of the method, the copper precursor may be copper oxide or a copper salt including copper nitrate, copper sulfate, copper chloride, copper bromide, copper fluoride, copper acetate, or a combination of any two or more thereof.

In some embodiments, the caustic material may include a sodium source. The sodium source may be sodium carbonate, sodium hydroxide or a combination thereof. In other embodiments, the caustic material may include a potassium source. The potassium source may be potassium carbonate or potassium hydroxide. In another embodiments, the caustic material may be sodium carbonate, sodium hydroxide, potassium carbonate, potassium hydroxide, or a combination of any two or more thereof.

The caustic material was added to the solution so that the pH of the aqueous slurry was about 7.0 to about 8.0. In one embodiment, the pH of the slurry was about 8.0. When the pH of the slurry was about 8.0, the precipitate may form a catalyst at a target particle size.

In some embodiments, the amount of precipitate in the slurry is at least about 10%. In some embodiments, the amount of precipitate in the slurry is at least about 15%, at least about 20 wt %, at least about 25 wt %, or at least about 30 wt %. In some embodiments, the amount of precipitate in the slurry is about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, or about 85 wt %.

Prior to activation, the catalyst may include about 30 wt % to about 40 wt % CuO, about 2 wt % to about 10 wt % NiO, and about 50 wt % to about 68 wt % Al₂O₃. In another embodiment, the catalyst may include about 30 wt % to about 40 wt % CuO, about 2 wt % to about 6 wt %, and the remaining be Al₂O₃.

In the method, the catalyst may be substantially free of chromium. In another embodiment of the method, the catalyst does not include chromium.

In the method, the BET surface area of the powder of the catalyst may be from about 20 m²/g to about 50 m²/g.

In another aspect, a method of hydrogenating/hydrogenolysis of a carbonyl-containing organic compound is provided. The method includes contacting the carbonyl-containing organic compound with an activated catalyst that is any of the catalysts described herein. In various embodiments, the carbonyl-containing organic compound may include a ketone, an aldehyde, and/or an ester. In some embodiments, it is a fatty acid ester. More specifically, in any embodiment disclosed herein, the carbonyl-containing organic compound may include, but is not limited to, a fatty acid methyl ester (e.g., C₈-C₂₀ carbon chain), fatty acid wax ester (e.g., C₁₈-C₄₀ carbon chain), furfural, methyl phenyl ketone, di-methyl or di-ethyl esters, or a mixture of any two or more thereof. The hydrogenation/hydrogenolysis may be carried out in a slurry phase reactor that may be a batch or semi-batch reactor, a continuously stirred tank reactor, a tower reactor, or a column reactor.

In another embodiment, the hydrogenation/hydrogenolysis may be carried out in a continuous process, where the catalyst may be recycled into the slurry phase reactor.

### EXAMPLES

Specific embodiments of the invention will now be demonstrated by reference to the following examples. It should be understood that these examples are disclosed solely by way of illustrating the invention and should not be taken in any way to limit the scope of the present invention.

A sense of copper (Cu), nickel (Ni), and aluminum (Al) catalysts were prepared having different ratios. The catalysts were fully characterized and evaluated for catalytic performance for fatty ester hydrogenolysis applications.

### Catalyst Preparation Procedure

A commercial copper chromium (“CuCr”) catalyst was used as a reference example. The CuCr catalyst included CuO at 46 wt %, Cr₂O₃ at 48 wt % and MnO at 6 wt %. The raw material used in Examples 1 to 4 is presented in Table 1.

TABLE 1

| Raw material | |
|---|---|
| Cu(NO₃)₂ | Copper nitrate, 17% |
| Al(NO₃)₃, 9H₂O | Aluminum nitrate, nonahydrate |
| Ni(NO₃)₂, 6H₂O | Nickel nitrate, hexahydrate |
| Na₂CO₃ | Sodium carbonate |
| NaOH | Pellet or 50% NaOH solution |

### Preparation of Example 1

Example 1 was prepared as follows to form Cu alumina with 6 wt % NiO. 282.4 g of copper nitrate solution was weighed into a beaker. 34.5 g of nickel (II) nitrate hexahydrate solid, and 622.1 g of aluminum nitrate nonahydrate were weighed and added into 250 g of deionized (DI) water. The mixture was warmed at 75° C. and stirred until it became clear. Once clear, this mixture was poured into the copper nitrate solution and mixed to form a CuNiAl solution. In a separate container, 132 g of sodium carbonate and 332 g of sodium hydroxide flakes were added along with 3977 g of DI water, and mixed until a clear solution formed.

A 2.5 L deionized water heel was placed in a baffled strike tank, i.e. precipitation vessel, with an agitation rate of 250 RPM. The CuNiAl solution was added to the precipitation vessel at a rate of 15 mL/min. The mixture of sodium carbonate and sodium hydroxide solution was added simultaneously to the precipitation vessel at a rate of about 45 mL/min, keeping the slurry at a constant pH of about 8.0. The pH was controlled by adjusting the rate of addition of the sodium carbonate and sodium hydroxide solution at room temperature.

The slurry was filtered to collect a filter cake solid. The filter cake was washed with DI water and dried at 120° C. overnight. From this preparation, the catalyst had the following chemical composition: 36% CuO-6% NiO-56% Al₂O₃.

After drying, the powder was calcined at 900° C. for four hours.

### Preparation of Example 2

Example 2 was prepared in a similar manner to Example 1, but less dilution was used. Example 2 was prepared to form Cu alumina with 6 wt % NiO. 282.4 g of copper nitrate solution was weighed into a beaker. 34.5 g of nickel (II)nitrate hexahydrate solid and 622.1 g of aluminum nitrate nonahydrate were weighed and added into 250 g of deionized (DI) water. The mixture was warmed at 75° C. and stirred until it became clear. Once clear, this mixture was poured into the copper nitrate solution and mixed to form a CuNiAl solution. In a separate container, 132 g of sodium carbonate and 663.4 g of 50% sodium hydroxide solution were added along with 3645 g of DI water, and mixed until a clear solution formed.

A 1.3 L deionized water heel was placed in a baffled strike tank, i.e. precipitation vessel, with an agitation rate of 250 RPM. The CuNiAl solution was added to the precipitation vessel at a rate of 14.2 mL/min. The sodium carbonate and sodium hydroxide solution was added simultaneously to the precipitation vessel at a rate of about 41 mL % min, keeping the slurry at a constant pH of about 8.0. The pH was controlled by adjusting the rate of addition of the sodium carbonate and sodium hydroxide solution at room temperature.

The slurry was filtered to collect a filter cake solid. The filter cake was washed with DI water and dried at 120° C. overnight. From this preparation, the catalyst had the following chemical composition: 37% CuO-6% NiO-56% Al₂O₃.

After drying, the powder was calcined at 900° C. for four hours.

### Preparation of Example 3

Example 3 was prepared in a similar manner to Example 2, but less NiO was present in the catalyst. Example 3 was prepared to form Cu alumina with 2 wt % NiO. 423.6 g of copper nitrate solution was weighed into a beaker. 18 g of nickel nitrate hexahydrate solid and 933.15 g of aluminum nitrate nonahydrate were weighed and added into 375 g of deionized (DI) water. The mixture was warmed at 75° C. and stirred until it became clear. Once clear, this mixture was poured into the copper nitrate solution and mixed to form a CuNiAl solution. In a separate container, 140.5 g of sodium carbonate and 706.5 g of 50% sodium hydroxide solution were added along with 3882 g of DI water, and mixed until a clear solution formed.

A 1 L deionized water heel was placed in a baffled strike tank, i.e. precipitation vessel, with an agitation rate of 250 RPM. The CuNiAl solution was added to the precipitation vessel at a rate of 14.2 mL/min. The sodium carbonate and sodium hydroxide solution was added simultaneously to the precipitation vessel at a rate of about 41 mL/min, keeping the slurry at a constant pH of about 8.0. The pH was controlled by adjusting the rate of addition of the sodium carbonate and sodium hydroxide solution at room temperature.

The slurry was filtered to collect a filter cake solid. The filter cake was washed with DI water and dried at 120° C. overnight. From this preparation, the catalyst had the following chemical composition: 37% CuO-2% NiO-55% Al₂O₃.

After drying, the powder was calcined at 900° C. for four hours.

### Preparation of Example 4

Example 4 was prepared in a similar manner to Example 1, but less dilution was used and the Cu alumina was prepared having 10 wt % NiO. 423.6 g of copper nitrate solution was weighed into a beaker. 93 g of nickel nitrate hexahydrate solid and 933.15 g of aluminum nitrate nonahydrate were weighed and added into 375 g of deionized (DI) water. The mixture was warmed at 75° C. and stirred until it became clear. Once clear, this mixture was poured into the copper nitrate solution and mixed to form a CuNiAl solution. In a separate container, 140.5 g of sodium carbonate and 706.5 g of 50% sodium hydroxide solution were added along with 3645 g of DI water, and mixed until a clear solution formed.

A 1 L deionized water heel was placed in a baffled strike tank, i.e. precipitation vessel, with an agitation rate of 250 RPM. The CuNiAl solution was added to the precipitation vessel at a rate of 14.2 mL/min. The sodium carbonate and sodium hydroxide solution was added simultaneously to the precipitation vessel at a rate of about 41 mL/min, keeping the slurry at a constant pH of about 8.0. The pH was controlled by adjusting the rate of addition of the sodium carbonate and sodium hydroxide solution at room temperature.

The slurry was filtered to collect a filter cake solid. The filter cake was washed with DI water and dried at 120° C. overnight. From this preparation, the catalyst had the following chemical composition: 34% CuO-10% NiO-53% Al₂O₃.

After drying, the powder was calcined at 900° C. for four hours.

### Catalyst Properties

### Particle Size Distribution During Precipitation

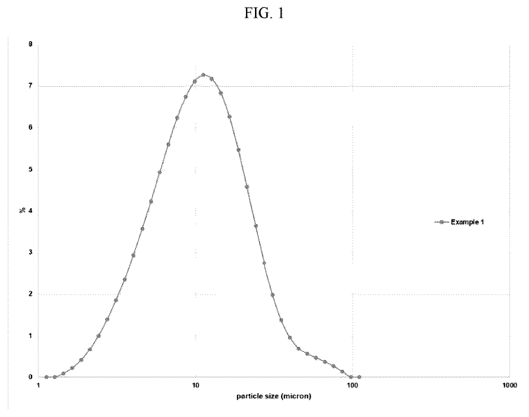

In FIG. 1, a graph is presented representing how the catalyst particle size is formed at the end of precipitation from Example 1. From the preparation in Example 1, the catalyst had a particle size distribution as follows:
D₁₀=4.4 microns
D₅₀=11.3 microns
D₉₀=27 microns

Among Examples 1-4, the average particle size distribution, D₅₀, was in the range of 7 to 12 microns depending on precipitation conditions.

### Chemical Composition of the Catalysts

The chemical composition of the catalysts prepared in Examples 1-4 are presented in Table 2.

TABLE 2
Chemical Composition of the Catalysts

| Example | CuO, wt % | NiO, wt % | Al₂O₃, wt % |
|---|---|---|---|
| 1 | 36 | 6 | 56 |
| 2 | 37 | 6 | 56 |
| 3 | 37 | 2 | 55 |
| 4 | 34 | 10 | 53 |

### BET Surface Area Measurement

The BET surface area measurement was performed following ASTM method D3663-03 Standard Test Method for Surface Area of Catalysts and Catalyst Carriers. The BET surface area of the catalysts of Examples 1-4 is shown in Table 3.

TABLE 3
BET Surface area (SA) of the calcined powder

| | Calcination | BET |
|---|---|---|
| Example | Temperature, ° C. | SA, m²/g |
| 1 | 900 | 26.5 |
| 2 | 900 | 40.1 |
| 3 | 900 | 24.7 |
| 4 | 900 | 24.6 |

### XRD Analysis of Example 2 to 4 Catalysts

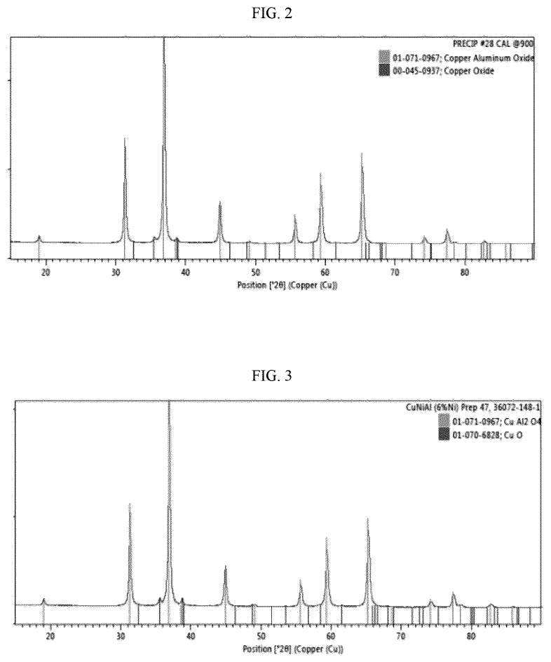

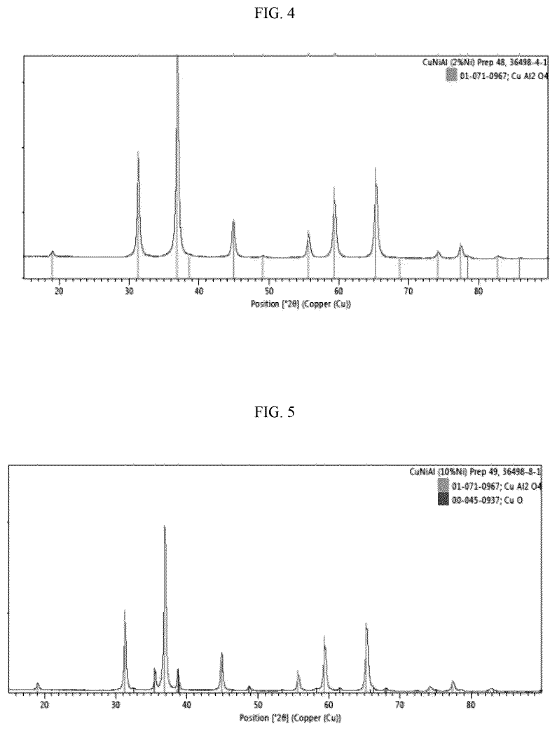

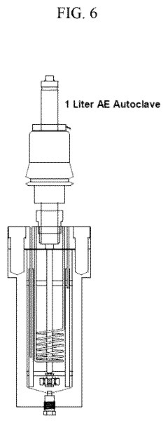

The current catalysts were calcined at 900° C. to get CuO and CuAl₂O₄ as the crystalline phases. The XRD images are presented in FIGS. 2-5. FIG. 2 represents the XRD pattern for Example 1. FIG. 3 represents the XRD pattern for Example 2. FIG. 4 represents the XRD pattern for Example 3. FIG. 5 represents the XRD pattern for Example 4.

It was found that NiO stabilized CuO and CuAl₂O₄ phases without sintering which resulted in a BET SA of 25-50 m²/g. As NiO loading was increased from 2 to 6 to 10 wt %, more CuO phase appeared along with CuAl₂O₄, which can be seen in FIGS. 2 to 5.

### Catalytic Performance

### Testing Procedure

Catalytic activity of the inventive catalysts of Examples 1-4 were evaluated by slurry phase hydrogenolysis of methyl ester to fatty alcohol. Catalyst performance evaluation was performed for both methyl ester hydrogenolysis and wax ester hydrogenolysis in a one-liter autoclave as shown in FIG. 6.

The procedure of methyl-ester hydrogenolysis testing is shown below:

### Reaction Conditions:

| | |
|---|---|
| Temperature, ° C. | 280 |
| Total pressure, psig | 2500 |
| Agitation Rate, rpm | 2000 |
| Catalyst Loading Level, wt. % | 0.8 |

### Feedstock Compositions:

| Compound | Amount % |
|---|---|
| C-8 ME | 0 |
| C-10 ME | 0.02 |
| C-12 ME | 74.83 |
| C-14 ME | 23.05 |
| C-16 ME | 0.57 |
| C-18:0 ME | 0 |
| C-18:1 ME | 0 |
| C-18:2 ME | 0 |
| C24 WE | 0.57 |
| 12:14 + 14:12 WE | 0.61 |

ME = methyl ester
WE = wax ester

### Procedure

The catalyst was loaded (0.8 wt % catalyst loading) by opening the top screw of the reactor head. 452 g of C12-C14 fatty acid methyl ester was loaded. The autoclave system was purged with N2 a few times to remove air and then the system was purged with hydrogen a few times.

Agitation was set to 2000 RPM and the temperature was ramped up using the furnace which jackets the autoclave body. The typical ramping rate of the temperature was 3° C./min. When the temperature reached 280° C., the autoclave was pressurized to 2500 psi, which relates to time “0.” Every hour, 5 mL of liquid was collected through a fritted port. This was done for 5 hours. The samples were analyzed via gas chromatography (GC) whereby fatty alcohol products, unreacted esters, and byproducts were quantified. The total fatty alcohol yield was calculated by summing the concentration of all fatty alcohol products.

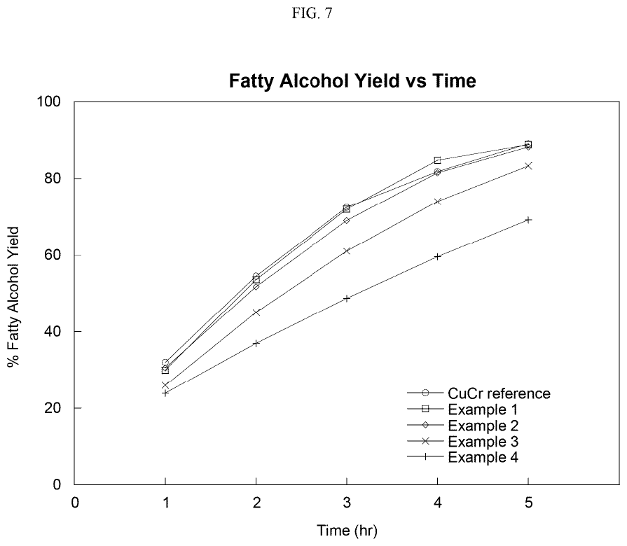

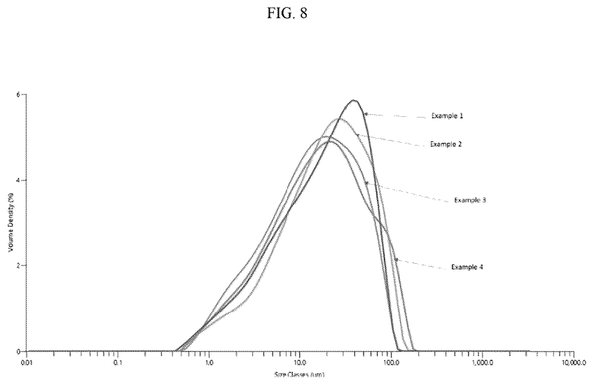

The performance of Examples 1-4 were compared to commercial CuCr catalysts and can be seen in FIG. 7. From FIG. 7, Examples 1 and 2, having 6 wt % of NiO) had comparable performance with the current commercial CuCr catalyst. The higher NiO loaded (10 wt %) catalyst of Example 4 is hypothesized to coat the Cu/alumina system, thus reducing the available active sites for reaction.

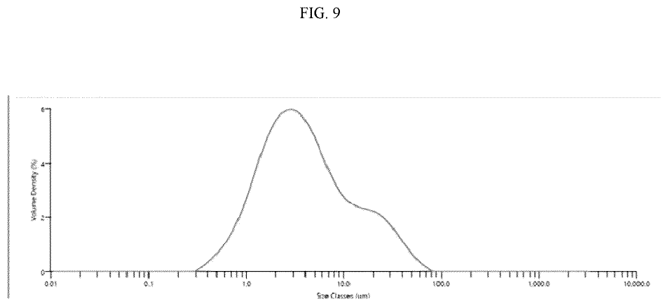

The particle size distribution of the fresh catalyst powder was also evaluated and presented in FIG. 8 and Table 4.

TABLE 4
Particle Size Distribution of the calcined powder

| | D₁₀(microns) | D₅₀(microns) | D₉₀(microns) |
|---|---|---|---|
| Example 1 | 2.9 | 19.3 | 59.4 |
| Example 2 | 3.6 | 20.6 | 69.8 |
| Example 3 | 2.5 | 14.8 | 54.3 |
| Example 4 | 2.8 | 17.1 | 74.3 |

The particle size distribution of Example 2 after reaction is shown in FIG. 9. In FIG. 9, it is important to note that after reaction, the catalyst powder did not disintegrate into very small fine particles. Too many small fine particles may slow down the separation process downstream of the reaction.

### Catalyst Filtration Properties

Catalyst separation experiments (both centrifuge separation and filtration) were conducted to compare the catalyst filterability. The results are summarized below and show that the inventive CuNiAl catalysts have comparable separation properties.

Procedure for centrifugation: A qualitative estimate on settling and separation by centrifugation at 11000 rpm for 5 minutes was performed. A picture was taken before and after for visual comparison. At first, 50 mL of the spent catalyst slurry with the liquid products and unreacted fatty acid methyl ester (0.8 wt % catalyst loading) was put in centrifuge for 5 minutes. Then, 35 mL of liquid was collected and a picture was taken to see if any particles were floating. The separation efficiency was measured qualitatively based on the color and fine particles floating in the collected liquid. The centrifuge separation test showed that the inventive catalysts and commercial CuCr had clear liquid product.

Procedure for Filtration: 12 mL of the used catalyst slurry (well mixed) was taken into a syringe and a syringe filter (0.45 μm) was then placed at the tip of the syringe. While being timed, the slurry was pressed through the syringe filter to separate the liquid products from the slurry. The time to complete the separation of the 12 mL sample was recorded. In the event filtrate could no longer be pushed out with maximum hand pressure, the timer was stopped and the filter changed. Timing of the process was then resumed until the separation was completed. Therefore, this test quantitatively estimates the ease of filtration by the number of filters required and time required for making the same amount of filtrate using a 0.45 μm filter. As can be seen in the table below, the inventive catalyst of Example 1 had a similar filtration time as the CuCr reference.

| Catalyst | Time to Collect | Number of filters |
|---|---|---|
| CuCr | 35 minutes | 1 |
| Example 1 | 40 minutes | 1 |

While certain embodiments have been illustrated and described, it should be understood that changes and modifications can be made therein in accordance with ordinary skill in the art without departing from the technology in its broader aspects as defined in the following claims.

The embodiments, illustratively described herein, may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of’ will be understood to include those elements specifically recited and those additional elements that do not materially affect the basic and novel characteristics of the claimed technology. The phrase “consisting of’ excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

1: A catalyst comprising:
a copper source;
a nickel source; and
alumina,
wherein the catalyst is substantially free of chromium.

2: The catalyst of claim 1, wherein the catalyst does not include chromium.

3: The catalyst of claim 1, wherein the catalyst has an average particle size of about 8 μm to about 12 μm.

4. (canceled)

5: The catalyst of claim 1, wherein the catalyst includes the copper source in an amount of about 30 wt % to about 40 wt % based on a total weight of the catalyst.

6: The catalyst of claim 1, wherein the catalyst includes the nickel source in an amount of about 2 wt % to about 6 wt % based on a total weight of the catalyst.

7: The catalyst of claim 1, wherein the catalyst has a Brunauer-Emmett-Teller (“BET”) surface area of about 20 m²/g to about 50 m²/g.

8: The catalyst of claim 1, wherein the copper source comprises copper oxide, copper nitrate, copper sulfate, copper chloride, copper bromide, copper fluoride, copper acetate, copper carbonate, or a combination of any two or more thereof.

9. (canceled)

10: The catalyst of claim 1, wherein the nickel source is nickel sulfate, nickel chloride, nickel bromide, nickel acetate, nickel oxide, nickel nitrate or a combination of any two or more thereof.

11. (canceled)

12: The catalyst of claim 1, wherein the alumina is aluminum nitrate, sodium aluminate or powder alumina.

13: A method of preparing a catalyst, comprising:
mixing a nickel precursor and alumina in a solution;
adding the solution to a copper precursor and mixing to form a second solution;
adding a caustic material to the second solution to form an aqueous slurry including a precipitate;
collecting the precipitate;
drying the precipitate to form a dried precipitate; and
calcining the dried precipitate to form a catalyst,
wherein the calcining is conducted at a temperature of about 850° C. to about 900° C., and wherein the catalyst has a Brunauer-Emmett-Teller (“BET”) surface area of about 15 m²/g to about 50 m²/g.

14: The method of claim 13, wherein the aqueous slurry is at a pH of about 7.0 to about 8.0.

15: The method of claim 13, wherein the collecting includes filtering the aqueous slurry to remove the precipitate.

16: The method of claim 13, wherein the calcining is conducted for about 1 hour to about 4 hours.

17: The method of claim 13, wherein the copper precursor is a copper salt including copper nitrate, copper sulfate, copper chloride, copper fluoride, copper bromide, copper acetate, or a combination of any two or more thereof.

18: The method of claim 13, wherein the nickel precursor includes nickel nitrate or nickel oxide.

19: The method of claim 13, wherein the alumina is aluminum nitrate, sodium aluminate or powder alumina.

20: The method of claim 13, wherein the caustic material is sodium carbonate, sodium hydroxide, potassium carbonate, potassium hydroxide, or a combination of any two or more thereof.

21: The method of claim 13, wherein the catalyst is substantially free of chromium.

22: The method of claim 13, wherein the catalyst does not include chromium.

23: A method of hydrogenating a carbonyl-containing organic compound, the method comprising contacting the carbonyl-containing organic compound with a catalyst of claim 1.
